(19) United States
(12) Patent Application Publication
(10) Pub. No.: US 2016/0175157 A1
(43) Pub. Date: Jun. 23, 2016

(54) Impregnable and Expanding Wound Packing Article

(71) Applicant: Terence W. Kolb, Fletcher, NC (US)

(72) Inventor: Terence W. Kolb, Fletcher, NC (US)

(21) Appl. No.: 14/978,181

(22) Filed: Dec. 22, 2015

Related U.S. Application Data
(60) Provisional application No. 62/095,216, filed on Dec. 22, 2014.

(51) Int. Cl. A61F 13/00; A61M 3/02

(52) U.S. Cl. CPC A61F 13/00068; A61F 13/00017; A61F 13/00063; A61F 13/00012; A61M 3/02

(57) ABSTRACT

An impregnable and expanding wound packing article includes a main body at least partially formed from a highly absorbent material, such as polyurethane sponge material or compressed cotton gauze sponge material. In one embodiment, the main body includes a component that is at least partially impregnable with a fluid element, such as a medicinal fluid. In operation, a care provider irrigates an open wound such that the main body may be engagedly received therein. In response to absorption of surrounding fluids, the main body continually expands until the outer wall of the main body comes into abutment with the inner wall of the irrigated, open wound. An elongated piece of material forming a wick extends from the main body and is sized and configured for grasping by a care provider in order to remove the expanded main body from the irrigated wound.

### BACKGROUND OF THE INVENTION

This application is based on and claims priority to provisional patent application No. 62/095,216 filed on Dec. 22, 2014.

### FIELD OF THE INVENTION

This invention relates to treatment of open wounds, such as abscesses, and, more particularly, to an impregnable and expanding article for packing open wounds.

### DISCUSSION OF THE RELATED ART

Presently used treatment methods for packing wounds have been in place for a number of years. An example of such a longstanding treatment method is the most common practice for treating an abscess, which generally includes the following steps: (1) Sterilizing the treatment site; (2) Applying a local anesthetic to the site; (3) Using a blade to incise the most fluctuant area of the abscess; (4) Using an instrument to express the purulent material contained within the tissue surrounding the abscess by breaking up the loculations; (5) Using forceps or a swab to repeatedly insert segments of one or more packing strips in order to fill the drained/irrigated abscess; (6) Covering the packed abscess with a bandage; (7) After approximately 48 hours, removing the packing strip(s) and repacking the abscess; and (8) Continuing the removal and repacking process until the site is no longer draining purulent material.

Steps 5, 7 and 8 listed above require use of one or more standard “shoestring-style” packing strips, which can require extensive, continuous packing in order to entirely fill a particularly sized abscess. Packing an abscess can be very painful for a patient and, because the above-described process requires repeated packing and unpacking of segments of the packing strip(s), the patient is forced to endure the associated pain a considerable number of times. A practical byproduct of this painful, segment-by-segment packing process is the likelihood that the care provider might pack the wound only to a suitable threshold rather than to completion due to the patient's discomfort. Improper packing procedure can result in outpatient therapy failure, thereby necessitating that the patient be admitted to the hospital. Moreover, because the above-described process requires an extended period of time for a physician to effectively complete, the associated medical costs for a patient are relatively high for what is otherwise a fairly routine procedure.

Other types of open wounds that often require packing include, but are not limited to: chronic non-healing ulcers; diabetic ulcers; superficial post-op surgical sites; bartholin gland cyst sites; and post-device (e.g., pacemakers) extraction openings.

Therefore, with the foregoing reasons in mind, there exists a need for an expanding article for packing an open wound, wherein the expanding article is selectively impregnable with one or more medicinal fluids.

### SUMMARY OF THE INVENTION

The present invention is directed to an impregnable and expanding wound packing article. The wound packing article includes a main body at least partially formed from a highly absorbent material, such as polyurethane sponge material or compressed cotton gauze sponge material. In one embodiment, the main body includes a component that is at least partially impregnable with a fluid element, such as a medicinal fluid. In operation, a care provider irrigates an open wound such that the main body may be engagedly received therein. In response to absorption of surrounding fluids, the main body continually expands until the outer wall of the main body comes into abutment with the inner wall of the irrigated, open wound. An elongated piece of material forming a wick extends from the main body and is sized and configured for grasping by a care provider in order to remove the expanded main body from the irrigated wound.

### BRIEF DESCRIPTION OF THE DRAWINGS

For a fuller understanding of the nature of the present invention, reference should be made to the following detailed description, taken in conjunction with the accompanying drawings in which:

FIG. 1 is a side elevational view illustrating the dermis layer, including the epidermis, dermis and hypodermis, muscle and bone;

FIG. 2 is a side elevational view illustrating a non-irrigated wound formed within the dermis layer;

FIG. 3 is a side elevational view of the wound packing article of the present invention according to one embodiment in an irrigated, open wound;

FIG. 4 is a side elevational view of the wound packing article according to the embodiment shown in FIG. 3 after it has expanded against the inner facing wall of the irrigated, open wound;

FIG. 5 is a side elevational view of the wound packing article of the present invention according to another embodiment in an irrigated, open wound; and

FIG. 6 is a side elevational view of the wound packing article according to the embodiment shown in FIG. 5 after it has expanded against the inner facing wall of the irrigated, open wound.

Like reference numerals refer to like reference parts throughout the several views of the drawings.

### DETAILED DESCRIPTION OF THE PREFERRED EMBODIMENT

Referring to the several views of the drawings, the impregnable and expanding wound packing article of the present invention for packing open wounds 100 is shown and is generally indicated as 10.

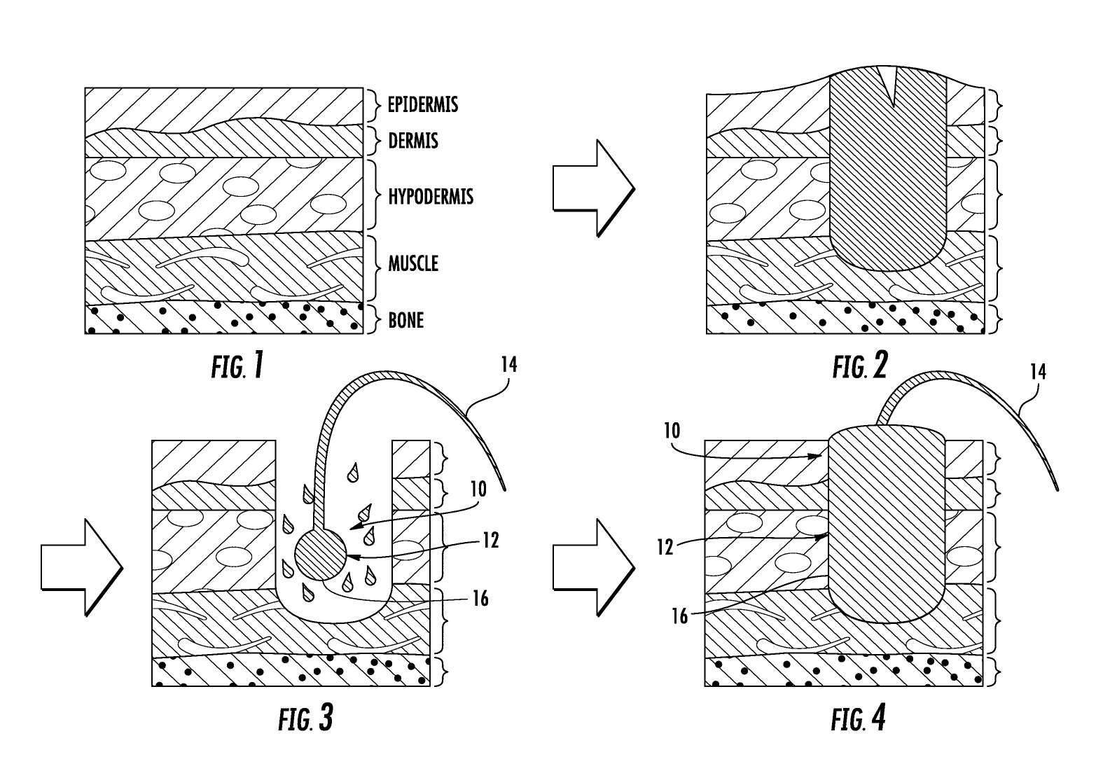

Referring initially to FIGS. 1-4, the wound packing article 10 includes a main body 12 and a wick 14 extending therefrom. The main body 12 is sized and configured for engaged receipt within an open wound 100, such as an irrigated abscess on the surface of a patient's skin. Different embodiments of the main body 12 may be formed having variously sized three-dimensional shapes (e.g., spherical, ovoidal, disk-shaped, etc.) and volumes. The wick 14 is an elongated piece of material that is secured to the main body 12 or, alternatively, formed integrally therefrom.

The main body 12 of the wound packing article 10 is at least partially made from one or more highly absorbent materials, such as polyurethane sponge material or compressed cotton gauze sponge material. The main body 12 may include an outer and/or inner layer of the absorbent material or, alternatively, may be made entirely from the one or more absorbent materials.

In a preferred embodiment, the main body 12 of the wound packing article 10 includes a component that is at least partially impregnable with a fluid element, such as a medicinal fluid (e.g., an antibiotic solution). The main body 12 is structured and disposed to emit the impregnated fluid element over a period of time when received within an open wound. In one embodiment, the emission of the impregnated element from the main body 12 is directly related to the absorption of bodily fluids by the main body 12.

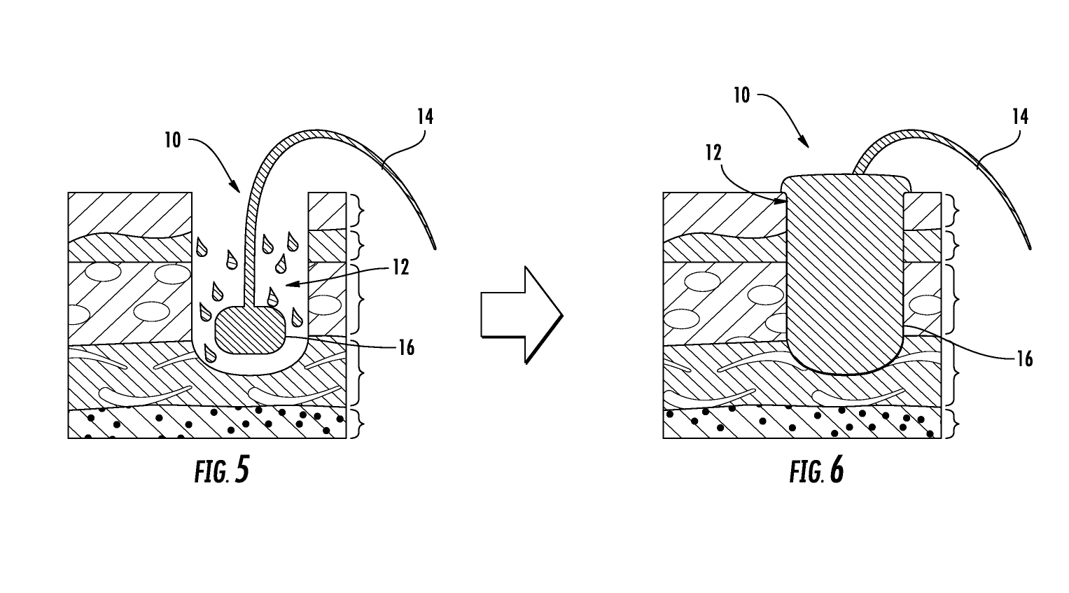

Referring specifically to FIGS. 3 and 4, the main body 12 of the wound packing article 10 is positioned within the irrigated, open wound 100 (e.g., irrigated abscess). A preferred method of introducing the main body 12 within the irrigated, open wound 100 is with forceps. FIG. 3 illustrates a first embodiment of the wound packing article 10, wherein the main body 12 is spherical. After the main body 12 is packed—which is a one step process—the wound is covered with a bandage. Over time, the wound cavity will fill with bodily fluids (e.g., purulent material) and the main body 12 absorbs the bodily fluids introduced thereto. In response to absorption of surrounding fluids, the main body continually expands until the outer wall of the main body 12 comes into abutment with the inner wall of the irrigated, open wound, as shown in FIG. 4. After approximately 48 hours, the expanded main body 12 is removed (one step process) from the irrigated, open wound 100 using the wick 14. The wound 100 may then be packed again with an unused, secondary wound packing article 10 if determined to be necessary by the care provider.

In a preferred embodiment, the outer wall 16 of the main body 12 is formed from a material having density sufficient for preventing surrounding tissue from growing into main body 12. In one embodiment, the gaps in the material forming the outer wall 16 are sufficiently narrow to prevent tissue growing therethrough.

Referring to FIGS. 5 and 6, another embodiment of the wound packing article 10 is shown, wherein the main body 12 is disk-shaped. The packing, absorption, expansion progression of the disk-shaped main body 12 is the same as described above for the spherical main body 12. Alternatively shaped main bodies 12 are considered and may be manufactured to suit particularly shaped open wounds.

In one embodiment of the wound packing article 10, the same material is used to form one or more of the highly absorbent, expanding and outer wall 16 portions of the main body 12.

While the present invention has been shown and described in accordance with several preferred and practical embodiments, it is recognized that departures from the instant disclosure are contemplated within the spirit and scope of the present invention.

What is claimed is:

1. An article for packing irrigated wounds, said article comprising:
a main body being at least partially formed from a highly absorbent material, said main body being sized and configured for engaging the irrigated wound;
said main body being structured and disposed for absorbing surrounding purulent fluid of the irrigated wound;
wherein absorption of the surrounding purulent fluid in the irrigated wound causes the main body to continually expand until the outer wall of said main body comes into abutment with the inner wall of the irrigated wound; and
an elongated piece of material defining a wick extending from the main body, said wick being sized and configured for grasping during removal of said expanded main body from the irrigated wound.

2. The article as recited in claim 1 wherein said highly absorbent material is a polyurethane sponge material.

3. The article as recited in claim 1 wherein said highly absorbent material is a compressed cotton gauze sponge material.

4. The article as recited in claim 1 wherein said main body is spherical-shaped.

5. The article as recited in claim 1 wherein said main body is disc-shaped.

6. The article as recited in claim 1 wherein said main body is ovoidal-shaped.

7. An article for packing irrigated wounds, said article comprising:
a main body being at least partially formed from a highly absorbent material, said main body being sized and configured for engaging the irrigated wound;
said main body being structured and disposed for absorbing surrounding purulent fluid of the irrigated wound;
wherein absorption of the surrounding purulent fluid in the irrigated wound causes the main body to continually expand until the outer wall of said main body comes into abutment with the inner wall of the irrigated wound;
one or more porous regions formed in said main body, and each of said one or more porous regions being in liquid flow communication with the outer wall of said main body; and
a fluid element that is impregnated in at least one of said one or more porous regions of said main body, said fluid element being exposed to the inner wall of the irrigated wound when the outer wall of said main body is in abutment with the inner wall of the irrigated wound.

8. The article as recited in claim 7 further comprising an elongated piece of material defining a wick extending from the main body, said wick being sized and configured for grasping during removal of said expanded main body from the irrigated wound.

9. The article as recited in claim 7 wherein said highly absorbent material is a polyurethane sponge material.

10. The article as recited in claim 7 wherein said highly absorbent material is a compressed cotton gauze sponge material.

11. The article as recited in claim 7 wherein said main body is spherical.

12. The article as recited in claim 7 wherein said main body is disc-shaped.

13. The article as recited in claim 7 wherein said main body is ovoidal-shaped.

14. The article as recited in claim 7 wherein said fluid element is medicinal fluid.

15. The article as recited in claim 14 wherein said fluid element is an antibiotic solution.

16. A method for packing an open wound, said method comprising the steps of:
irrigating the open wound;
engaging the irrigated, open wound with a wound packing article, said wound packing article comprising:
a main body being at least partially formed from a highly absorbent material, said main body being sized and configured for engaging the irrigated wound;
said main body being structured and disposed for absorbing surrounding purulent fluid of the irrigated wound;
wherein absorption of the surrounding purulent fluid in the irrigated wound causes the main body to continually expand until the outer wall of said main body comes into abutment with the inner wall of the irrigated wound; and
an elongated piece of material defining a wick extending from the main body, said wick being sized and configured for grasping during removal of said expanded main body from the irrigated wound;
waiting a predetermined period of time; and
removing said wound packing article from said irrigated, open wound.

17. The method for packing an open wound as recited in claim 16 comprising the additional steps of:
engaging the irrigated, open wound with a secondary wound packing article, said secondary wound packing article comprising:
a main body being at least partially formed from a highly absorbent material, said main body being sized and configured for engaging the irrigated wound;
said main body being structured and disposed for absorbing surrounding purulent fluid of the irrigated wound;
wherein absorption of the surrounding purulent fluid in the irrigated wound causes the main body to continually expand until the outer wall of said main body comes into abutment with the inner wall of the irrigated wound; and
an elongated piece of material defining a wick extending from the main body, said wick being sized and configured for grasping during removal of said expanded main body from the irrigated wound;
waiting a predetermined period of time; and
removing said secondary wound packing article from said irrigated, open wound.
